(19) United States
(12) Patent Application Publication
(10) Pub. No.: US 2010/0305664 A1
(43) Pub. Date: Dec. 2, 2010

(54) Methods and Devices for Adrenal Stimulation

(76) Inventors: Brett M. Wingeier, San Francisco, CA (US); Benjamin David Pless, Atherton, CA (US); Anthony V. Caparso, San Jose, CA (US); Margaret McLaughlin, Hillsborough, CA (US)

Correspondence Address: SHAY GLENN LLP, 2755 CAMPUS DRIVE, SUITE 210, SAN MATEO, CA, 94403, US

(21) Appl. No.: 12/791,690

(22) Filed: Jun. 1, 2010

Related U.S. Application Data
(60) Provisional application No. 61/182,935, filed on Jun. 1, 2009.

(51) Int. Cl. A61N 1/36

(52) U.S. Cl. 607/62; 607/2

(57) ABSTRACT

An implantable medical device is provided for the treatment of a variety of disorders. The implantable medical device can be a neurostimulator having a stimulation lead and electrode(s) configured to be implanted on or near neural tissue in communication with the adrenal gland. Application of an electrical waveform to the neural tissue can cause the adrenal gland to release catecholamines to treat hypoglycemia. In other embodiments, chemical, magnetic, optical, or mechanical neuromodulation can be used.

### CROSS-REFERENCE TO RELATED APPLICATIONS

This application claims the benefit under 35 U.S.C. 119 of U.S. Provisional Patent Application No. 61/182,935, filed Jun. 1, 2009, titled “Methods and Devices for Adrenal Stimulation for Metabolic Disorders.” This application is herein incorporated by reference in its entirety.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD OF THE INVENTION

The present invention relates generally to an apparatus and method for delivering a therapeutic device to the adrenal glands of a subject for the treatment of metabolic disorders.

### BACKGROUND OF THE INVENTION

The adrenal glands or suprarenal glands are paired endocrine organs situated superior to the kidneys. Each adrenal gland consists of two distinct endocrine organs, the cortex and the medulla. The right gland is somewhat triangular in shape and the left is more semilunar, usually larger and placed at a higher level than the right. They vary in size in different individuals; however their usual size is from 4-6 cm in length, usually 2-3 cm in width and 0.2-0.6 cm thick. The adrenal glands are supplied by multiple and variable arteries that derive from the aorta, inferior phrenic and renal arteries. The suprarenal vein returns the blood from the medullary venous plexus and receives several branches from the adrenal cortex. The suprarenal vein opens on the right side into the inferior vena cava, on the left side into the renal vein. Most of the neural innervation of the adrenal glands is via the celiac plexus, splanchnic nerves and other abdominal ganglia, such as the mesenteric and aorticorenal. The splanchnic nerves originate from cells in the intermediolateral cell column of the thoracic spinal column. The splanchnic nerve innervation to the adrenal glands comes via the greater, lesser and least splanchnic nerves.

The adrenal medulla is located centrally within the adrenal gland, and plays a significant role in autonomic function. Chromaffin cells located in the adrenal medulla release catecholamines (CAs) such as epinephrine, norepinephrine, and dopamine into the bloodstream. The adrenal medulla is innervated largely by preganglionic sympathetic fibers of the greater, lesser and least splanchnic nerves, which originate in the thoracic spinal cord. These fibers synapse cholinergically (release acetylcholine as the neurotransmitter) upon the chromaffin cells and trigger CA release. The adrenal chromaffin cells release CAs directly into the circulating blood, and the CAs are carried in the blood to all tissues of the body. Circulating CAs result almost in the same physiological effect associated with sympathetic (“flight or fight”) response, such as increased heart rate, increased blood pressure, increased energy expenditure, increased glycogen breakdown, and bronchodilation, except the effects can last 5 to 10 times as long because these hormones are removed from the blood slowly.

Electrical stimulation of the splanchnic nerves is known to cause CA release. The CA composition of the adrenal gland effluents obtained during peripheral splanchnic nerve stimulation may be altered by changes in the stimulation frequency. At relatively high frequency (20 Hz), compared to the intrinsic autonomic frequencies, higher amounts of adrenaline are released (Mirkin 1961). The autonomic nervous system operates at a very low intrinsic frequency. Guyton (Guyton and Hall 2006) suggests that the autonomic nervous system only needs one nerve impulse every few seconds to maintain normal sympathetic and parasympathetic effects, and full activation occurs when the nerve fibers discharge 10 to 20 times per second (Guyton and Hall 2006). This differential secretion of catecholamines, elicited by different patterns of splanchnic nerve stimulation has also been corroborated by others (Klevans and Gebber 1970; Edwards and Jones 1993). Stimulation applied to structures of the sympathetic nervous system, such as the sympathetic chain ganglia, splanchnic nerves, celiac ganglia, or mesenteric ganglia, has been suggested for treatment of obesity (U.S. Pat. No. 7,239,912 to Dobak) via multiple mechanisms, including increase in resting energy expenditure due to CA release. Transmural stimulation of the surgically removed adrenal gland—that is, stimulation applied across the outer walls of the gland—is known to cause CA release (Wakade 1981; Alamo, Garcia et al. 1991). Finally, perfusion of the adrenal gland with acetylcholine (ACh) has also been shown to cause CA release (Wakade 1981).

The adrenal glands are positioned in the retroperitoneal space, immediately superior to the kidneys. The glands are relatively fragile. Open and laparoscopic surgical approaches, both transperitoneal and retroperitoneal, are well-known (Bonjer, Sorm et al. 2000); open approaches are significantly invasive. The adrenal medulla is highly vascular, with a complex arterial supply passing through the adrenal cortex, and a relatively simpler return through the adrenal medulla (Coupland and Selby 1976). Return is via the right suprarenal vein, which drains into the inferior vena cava, and the left suprarenal vein, which drains into the left renal vein or left inferior phrenic vein. Access via catheter to the suprarenal veins is well-known (Daunt 2005).

Diabetes mellitus, often referred to as diabetes, is a condition in which a person has a high blood sugar level, either because the body doesn't produce enough insulin, or because the body cells don't properly respond to insulin that is produce. Insulin is a hormone produced in the pancreas which enables body cells to absorb glucose, to turn to energy. Insulin is needed to regulate the amount of sugar in cells. Because the sugars are not being absorbed by the cells, cells are unable to operate as efficiently. Cellular functions rely on glucose sugar as their main source of energy. If diabetes is left unchecked, it can lead to stroke, cardiovascular disorders, blindness, kidney failure, amputations and nerve damage. In severe cases, diabetes can cause individuals to fall into a diabetic coma, known as diabetic ketoacidosis.

There are three types of diabetes which affect 18.2 million Americans. Type 1 diabetes is an autoimmune disease in which the body's immune system turn against its own cells. The insulin producing beta cells in the pancreas are attacked, causing insulin to be produced at an inefficient rate or not at all. Type II diabetes is the most common form of the disease; it is associated with being overweight, inactivity, older age, history of gestation diseases, ethnicity, and family history. Insulin is produced normally at first, but the body's cells become resistant to it and do not use the insulin correctly. Eventually, insulin production will decrease as cells become resistant. Type III diabetes is known as gestational diabetes and only occurs during pregnancy. It is thought to be caused by increased hormone levels, which create an insulin resistance similar to that found in Type II diabetes.

Individuals with diabetes are not able to automatically maintain their blood glucose levels within a safe physiological range. They are taught how to compensate by monitoring their blood glucose levels and by taking action if measured blood glucose levels fall outside of acceptable bounds or the levels are trending such that it can be predicted that the blood glucose will fall out of acceptable bounds. If a diabetic finds that the glucose level is too low he or she will eat an appropriate snack to raise their glucose level. If glucose levels are high or expected to rise for example after eating, people with diabetes have to take into account variables such as the caloric value of the meal they are eating and then take a suitable dose of insulin to keep their glucose level from increasing too much. If they take too much insulin, glucose levels can fall dangerously low, causing hypoglycemia. Likewise some diabetics do not have any symptoms when their glucose levels fall so they can be hypoglycemic without being aware of it and therefore cannot take suitable action.

Hypoglycemia refers to a lower than normal amount of glucose in the blood. Usually, the condition is mild and can be treated by the intake of sugar, but in chronic cases, the brain will not receive enough glucose, resulting in impaired function. Impaired function can lead to permanent brain damage or death if left untreated. Symptoms include shakiness, anxiety, nervousness, tremor, palpitations, sweating, hunger, nausea, fatigue and personality changes. Hypoglycemia can occur at any age and from a variety of causes, however, it commonly results as a complication from diabetes.

This invention uses a device and methods for indirect or direct stimulation of the adrenal gland, causing the release of catecholamines into the bloodstream. More specifically, the invention relates to the stimulation of the adrenal medulla to cause the secretion of epinephrine and other hormones that then cause glucose release from the liver to avoid hypoglycemia. In this invention, the control of the stimulation can be either open loop or closed loop.

### SUMMARY OF THE INVENTION

The present invention relates to a method of treating a disorder, comprising placing a stimulation lead on neural tissue in communication with an adrenal gland, and delivering an electrical waveform from a neurostimulator to the stimulation lead to release catecholamines from the adrenal gland to treat hypoglycemia.

In some embodiments, the stimulation lead is placed on or in an inferior vena cava. In other embodiments, the stimulation lead is placed on or in a left or right renal vein. In additional embodiments, the stimulation lead is placed on or in a left or right suprarenal vein. In another embodiment, the stimulation lead is placed on or in an inferior phrenic vein. In another embodiment, the stimulation lead is placed on or near a thoracic sympathetic trunk. In one embodiment, the stimulation lead is placed on or near a splanchnic nerve.

In some embodiments, the stimulation lead comprises a coiled shape. The stimulation lead can also comprise a stent shape, a sac, a net, or a cuff. In many embodiments, the stimulation lead comprises at least one electrode.

In some embodiments, the neurostimulator is implanted in a lower abdomen of the patient. The stimulation lead can be tunneled from a venous access site to the neurostimulator, for example. In one embodiment, the neurostimulator is implanted at a site of venous access. In another embodiment, the neurostimulator is implanted within a vessel.

In some embodiments of the method, the delivering step further comprises delivering the electrical waveform from the neurostimulator to the stimulation lead by positioning an external controller in close proximity to the neurostimulator.

In one embodiment, the stimulation lead comprises a neural cuff configured to stimulate pre-synaptic sympathetic nerves innervating the adrenal gland. The neural cuff can be approximately 12 to 25 mm in length, for example. In some embodiments, the neural cuff has an internal diameter that corresponds with an external diameter of a renal artery. In another embodiment, the neural cuff comprises electrodes that extend along an inner circumference for at least 270 degrees.

In another embodiment, the stimulation lead is placed around an adrenal cortex and is configured to stimulate adrenal medulla chromaffin cells. The stimulation lead can have a geometry resembling a Y and can comprise at least one electrode. In one embodiment, the stimulation lead comprises penetrating elements configured to penetrate the cortex of the adrenal gland. In another embodiment, the stimulation lead comprises a net configured to at least partially surround the adrenal gland. In one embodiment, the net comprises a plurality of flexible spines configured to allow the net to retain its shape upon deployment around the adrenal gland. In another embodiment, the net further comprises a drawstring configured to secure the net around the adrenal gland. The net can include at least one electrode for stimulation of the adrenal gland, for example.

Another method of treating a disorder in a patient is provided, comprising placing a stimulation lead on neural tissue in communication with an adrenal gland, measuring a glucose level of the patient with a continuous glucose monitor, and automatically applying an electrical waveform from the stimulation lead to the neural tissue when the glucose level is low to release catecholamines from the adrenal gland to treat hypoglycemia.

In some embodiments of the method, the stimulation lead is placed on or in an inferior vena cava. In other embodiments, the stimulation lead is placed on or in a left or right renal vein. In additional embodiments, the stimulation lead is placed on or in a left or right suprarenal vein. In another embodiment, the stimulation lead is placed on or in an inferior phrenic vein. In another embodiment, the stimulation lead is placed on or near a thoracic sympathetic trunk. In one embodiment, the stimulation lead is placed on or near a splanchnic nerve.

In some embodiments, the stimulation lead comprises a coiled shape. The stimulation lead can also comprise a stent shape, a sac, a net, or a cuff. In many embodiments, the stimulation lead comprises at least one electrode.

In some embodiments, the neurostimulator is implanted in a lower abdomen of the patient. The stimulation lead can be tunneled from a venous access site to the neurostimulator, for example. In one embodiment, the neurostimulator is implanted at a site of venous access. In another embodiment, the neurostimulator is implanted within a vessel.

In some embodiments of the method, the delivering step further comprises delivering the electrical waveform from the neurostimulator to the stimulation lead by positioning an external controller in close proximity to the neurostimulator.

In one embodiment, the stimulation lead comprises a neural cuff configured to stimulate pre-synaptic sympathetic nerves innervating the adrenal gland. The neural cuff can be approximately 12 to 25 mm in length, for example. In some embodiments, the neural cuff has an internal diameter that corresponds with an external diameter of a renal artery. In another embodiment, the neural cuff comprises electrodes that extend along an inner circumference for at least 270 degrees.

In another embodiment, the stimulation lead is placed around an adrenal cortex and is configured to stimulate adrenal medulla chromaffin cells. The stimulation lead can have a geometry resembling a Y and can comprise at least one electrode. In one embodiment, the stimulation lead comprises penetrating elements configured to penetrate the cortex of the adrenal gland. In another embodiment, the stimulation lead comprises a net configured to at least partially surround the adrenal gland. In one embodiment, the net comprises a plurality of flexible spines configured to allow the net to retain its shape upon deployment around the adrenal gland. In another embodiment, the net further comprises a drawstring configured to secure the net around the adrenal gland. The net can include at least one electrode for stimulation of the adrenal gland, for example.

The method can further comprise the step of monitoring a heart rate of the patient with a cardiac monitor. In some embodiments, the electrical waveform is automatically applied based on the glucose level and the monitored heart rate of the patient.

A system for stimulating an adrenal gland is also provided, comprising a continuous glucose monitor configured to measure a patient glucose level, a stimulation lead, and an adrenal stimulator in communication with the continuous glucose monitor and the stimulation lead, the adrenal stimulator configured to apply an electrical waveform to the stimulation lead in response to the measured patient glucose level.

In some embodiments, the system can comprise a cardiac monitor configured to measure a cardiac parameter.

In other embodiments, the stimulation lead is sized and configured to be implanted on neural tissue in communication with an adrenal gland. The stimulation lead can comprise a coiled shape, a stent shape, a net, a sac, a neural cuff, or a Y shape, for example. In many embodiments, the stimulation lead comprises at least one electrode. In some embodiments, the adrenal stimulator is in radio communication with the continuous glucose monitor.

### BRIEF DESCRIPTION OF THE DRAWINGS

FIG. 1 illustrates the general vascular and neural anatomy of the adrenal glands.

FIGS. 2a-2b show the close up vascular and neural anatomy of both the left and right adrenal glands.

FIG. 3 shows the possible locations of an adrenal neurostimulation lead placed intravascularly.

FIG. 4 is one embodiment of an adrenal neurostimulator placed intravascularly.

FIG. 5 is one embodiment of an adrenal stimulation device placed subcutaneously.

FIGS. 6a-6c show different embodiments of the distal portion of the stimulation lead.

FIG. 7 is one embodiment of the system including the adrenal stimulator, CGM and a heart rate monitor.

FIG. 8 is one embodiment of a small externally powered adrenal neurostimulator implanted subcutaneously.

FIG. 9 is a block diagram of one embodiment of the neurostimulator.

FIG. 10 is a block diagram of one embodiment of an external controller.

FIG. 11 is one embodiment of a neural cuff lead.

FIG. 12 is one embodiment of the sac lead.

### DETAILED DESCRIPTION OF THE INVENTION

The autonomic nervous system, which innervates numerous pathways within the human body, consists of two divisions: the sympathetic and parasympathetic nervous system. The sympathetic nervous system usually initiates activity within the body, preparing the body for action, while the parasympathetic nervous system primarily counteracts the effects of the sympathetic system.

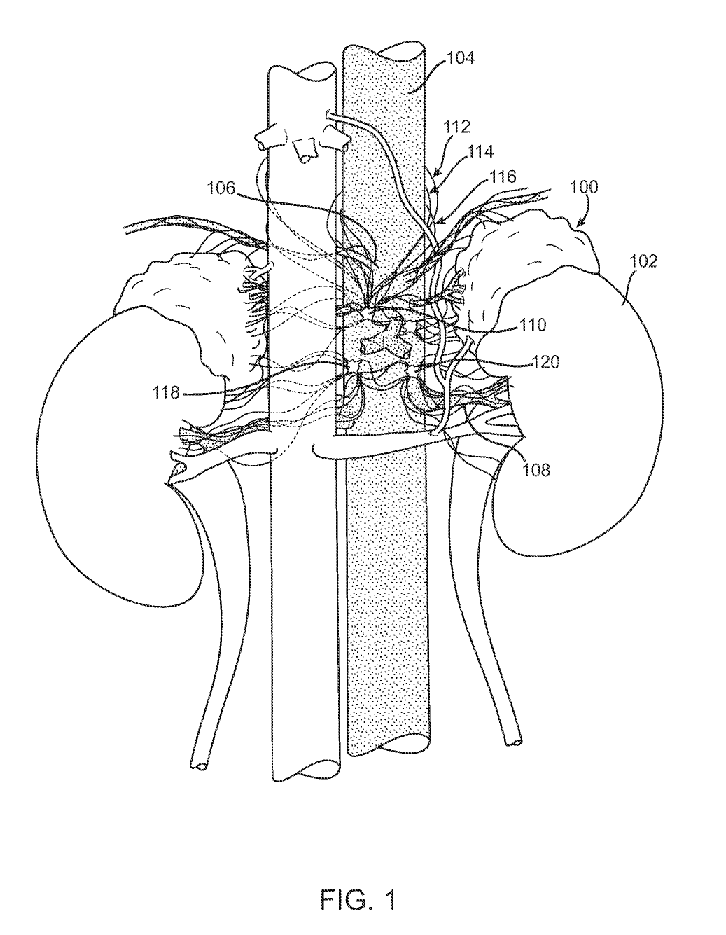

FIG. 1 shows the general anatomical, neural and vascular anatomy of the adrenal glands 100, which are located superior to the kidneys 102. Each adrenal gland is supplied by multiple and variable arteries that derive from the aorta 104, inferior phrenic 106 and renal arteries 108. The neural innervation of the adrenal glands 100 is via the celiac plexus and ganglia 110, splanchnic nerves; greater 112, lesser 114 and least 116, and other abdominal ganglia, such as the mesenteric 118 and aorticorenal 120. The adrenal medulla is innervated largely by preganglionic sympathetic fibers of the greater, lesser, and least splanchnic nerves, which originate in the thoracic spinal cord. These fibers synapse cholinergically upon the chromaffin cells and trigger CA release. Note that for simplicity, anatomical terms such as medulla or gland will be used in the singular, but the inventions described here may also be applied to both medullae at once. Also note that terms like the splanchnic nerves (greater, lesser and least) may be used in the singular, but may describe both sets of splanchnic nerves. Additionally, the terms celiac, mesenteric and aorticorenal ganglia may be referred to in singular, but may describe multiple ganglia as well.

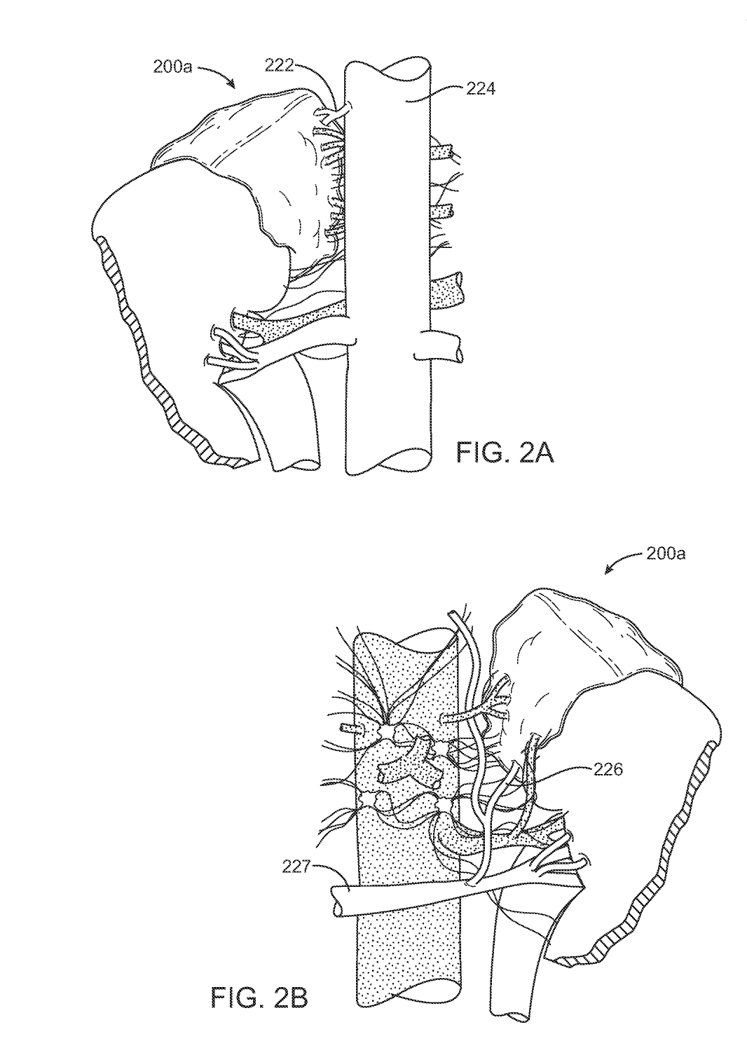

FIGS. 2a-2b show the detailed vascular supply to the adrenal glands, including to the right adrenal gland 200a (FIG. 2a) and to the left adrenal gland 200b (FIG. 2b). The right adrenal gland's vascular return is via the right suprarenal vein 222, which opens directly to the inferior vena cava 224. On the left side the venous return is via the left suprarenal vein 226, which drains to the inferior vena cava via the left renal vein 227.

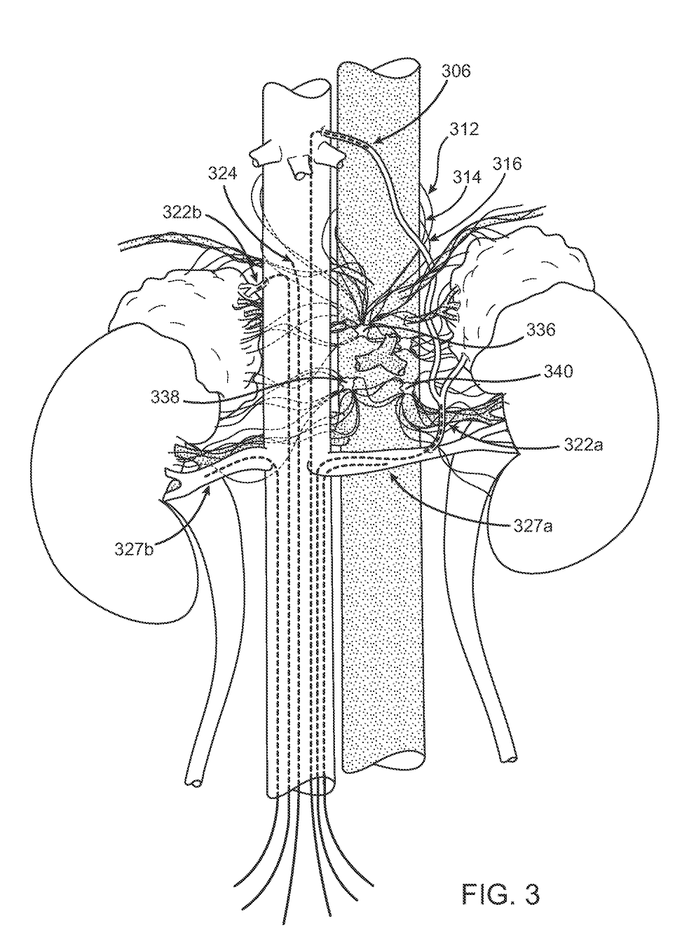

Stimulation of the adrenal medulla to cause the release of CAs may be accomplished in several ways. The adrenal medulla may be directly or indirectly stimulated by electrical waveforms or other forms of neuromodulation, including but not limited to chemical, magnetic, optical, mechanical (including vibration) or a combination of two or more of these. Referring to FIG. 3, stimulation of the adrenal medulla may be done through the activation of pre-ganglionic fibers that innervate the adrenal medulla prior to synapsing onto chromaffin cells. These fibers include but are not limited to the greater 312, lesser 314 and least 316 splanchnic nerve, (greater, lesser, least, or all including neuromodulation at the point of entry of preganglionic fibers into the adrenal gland and neuromodulation at the point where the sympathetic fibers exit the spinal cord, and including neuromodulation at the celiac 336, mesenteric 338 or aorticorenal 340 ganglion). Stimulation of the adrenal medulla or the pre-ganglionic neural fibers may also be done by placing a transvascular stimulation lead containing one or more electrodes within (for example, but not limited to) the inferior vena cava 324, left 327a or right 327b renal vein, inferior phrenic vein 306, left 322a or right 322b suprarenal vein, or any combination of these. In addition, electrical stimulation may be applied to the adrenal medulla via one or more electrodes applied to the outer capsule of the adrenal cortex; one or more electrodes inserted in the parenchyma of the adrenal medulla; one or more electrodes applied to the or encircling the suprarenal vein; one or more electrodes inserted at least partially into the medulla via the lumen of the suprarenal vein, or any combination thereof.

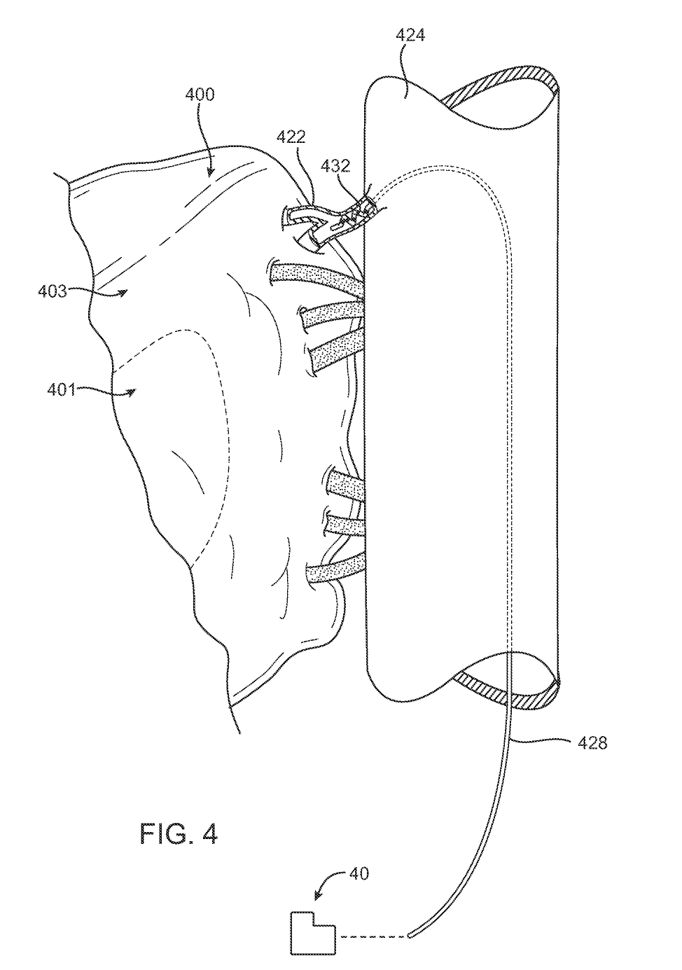

FIG. 4 illustrates one embodiment of an adrenal medulla stimulation device 40. The right adrenal gland 400, including the adrenal medulla 401 and the adrenal cortex 403, is shown in schematic view. The suprarenal vein 422 extends from the adrenal medulla 401 to the inferior vena cava 424. A stimulation lead 428 can be placed within the lumen of the suprarenal vein 422 or at least partially within the adrenal gland 400. Electrical stimulation can be delivered through one or more electrodes 432 located on the stimulation lead 428.

As shown in FIG. 4, electrical stimulation of the adrenal medulla can be accomplished by applying an electrical waveform from a neurostimulator to one or more electrodes of a transvascular lead placed within the lumen of the suprarenal vein. The electrical waveform delivered by the neurostimulator through one or more electrodes causes activation of the neural tissue and or adrenal chromaffin cells surrounding the lumen of the vessel. In this embodiment, the transvascular lead may have up to 16 electrodes positioned around the lumen of the vessel using a coiled lead geometry as shown in FIG. 4. Each electrode when activated causes activation of a somewhat different population of neural fibers leading to the adrenal gland, which along with changes in stimulation frequency can change the catecholamine concentration secreted into the blood stream by the adrenal gland, referred to as the norepinephrine: epinephrine ratio. For example, if one population of neural fibers is activated they may cause the preferential release of norepinephrine over epinephrine and if another population of neural fibers are activated, they may cause the release of epinephrine predominately. Or, if one set of neural fibers are activated, using one set of stimulation parameters, it may cause the preferential release of norepinephrine over epinephrine and if the same set of neural fibers are activated, using a different set of stimulation parameters, it may cause the preferential release of epinephrine over norepinephrine.

For example, a physician using standard physiological monitoring, including but not limited to monitoring heart rate, blood pressure, airway resistance, pupil diameter, etc, can test the physiological response to indirect or direct stimulation of the adrenal gland. Norepinephrine and epinephrine, both of which are secreted into the blood by the adrenal medulla, have somewhat different effects in exciting the alpha and beta receptors. Norepinephrine excites mainly alpha receptors but also excites to a less extent beta receptors as well. Epinephrine, on the other hand, excites both types of receptors approximately equally. Therefore, the relative effects of norepinephrine and epinephrine on different effector organs are determined by the types of receptors in the organs. For example, alpha receptors found on peripheral blood vessels when activated by norepinephrine cause vasoconstriction and alpha receptors located on the iris when activated by norepinephrine cause iris dilatation. Beta receptors likewise will cause peripheral vasodilatation on blood vessels, acceleration of heart rate, increased myocardial strength and iris constriction when activated by epinephrine. Thus, having multiple electrodes, electrode configurations and the flexibility in the stimulation parameter settings a physician can determine the relative norepinephrine:epinephrine ratio released due to indirect or direct stimulation of the adrenal gland.

Indirect or direct stimulation of the adrenal gland via the greater, lesser, least, or all including neuromodulation at the point of entry of preganglionic fibers into the adrenal gland or neuromodulation at the point where the sympathetic fibers exit the spinal cord or stimulation of the sympathetic track within the spinal cord, and including neuromodulation at the celiac, mesenteric or aorticorenal ganglion is done using one or more electrodes. In one embodiment, using one set of electrodes and stimulation parameters, for example, continuous stimulation at 4 Hz at an amplitude and pulse width know to cause electrical activation of the neural tissue or adrenal tissue. This type of stimulation is known to cause preferential release of epinephrine over norepinephrine in animal's models including, calves, sheep, cats and rats (Edwards and Jones 1993). In other embodiments, using the same set of electrodes and a different set of stimulation parameters, for example, stimulation at 40 Hz for 1 second, at 10 second intervals at an amplitude and pulse width know to cause electrical activation of the neural tissue or adrenal tissue. This type of stimulation is know to cause preferential release of norepinephrine over epinephrine in animal's models including, calves, sheep, cats and rats (Edwards and Jones 1993). The same type of response may be accomplished through changing the electrode configuration or changing the electrodes used for the indirect or direct stimulation of the adrenal glands.

In another embodiment, the stimulation and electrode configuration for indirect or direct stimulation of the adrenal gland may not be critical in determining the relative norepinephrine:epinephrine ratio, as the physiological state of the patient. In this embodiment, in which the patient is experiencing a hypoglycemic event, stimulation of the adrenal gland can cause preferential release of epinephrine regardless of the electrode configuration or the stimulation parameters used. Stimulation of the adrenal gland during hypoglycemia is known to cause preferential release of epinephrine in cats (Duner, 1954). For hypoglycemia, releasing more epinephrine than norepinephrine will typically be most effective.

In the embodiment of FIG. 4, the stimulation lead 428 of the adrenal medulla stimulation device 40 can be placed at least partially within the suprarenal vein 422 via a transvascular system that comprises a standard introducer catheter that is inserted percutaneously into the femoral vein, and a guide wire. After gaining percutaneous access to the femoral vein, a small flexible guide wire is inserted into the introducer catheter and advanced up the femoral vein and into the inferior vena cava. Advancement of the guide wire is done using image guidance, e.g. fluoroscopy, and venography, which uses intravenous contrast agents such as iodine to understand the venous anatomy and help advance the guide wire. The guide wire is then advanced from the femoral vein into the inferior vena cava and then into the right suprarenal vein. Once the guide wire is place within the suprarenal vein the transvascular lead is then placed using the guide wire. The transvascular lead has a central lumen that is sized such that the transvascular lead can be advanced over the guide wire and into the intended position.

In other embodiments, advancement of the guide wire can be aided by using a series of flexible catheters. In one such embodiment, a more rigid guide wire is placed through the standard femoral vein introducer and advanced up to the inferior vena cava at the level of the kidney. Then a flexible catheter is introduced over the guide wire and advanced to the same level as the guide wire and the guide wire removed. A second more flexible guide wire is then advanced through the catheter and exits the catheter at the level of kidney. The flexible guide wire can then be steered into the suprarenal vein. The second more flexible guide wire may also have a very flexible and loose distal tip that is also steerable from the proximal end of the guide wire. Using intravenous contrast, the guide wire can be advanced into the suprarenal vein. The contrast solution can be delivered through a second working port on the proximal end of the flexible catheter, thus one port is for advancing the guide wire and the second for injecting the contrast solution for the venography. In this embodiment, the lead is again advanced over the guide wire into the intended target anatomy.

In one embodiment, the transvascular lead has a distal geometry that is configured to have the shape of a coiled spring in its native state. The distal portion of the lead changes its geometry when placed over the flexible guide wire such that it takes a linear (straight) geometry. When the transvascular lead is placed in situ and the guide wire is retracted the distal portion of the lead rebounds to its native geometry, a coiled spring, thus placing one or more electrodes in tight junction with the vessel wall in a 360 degree fashion. In this embodiment, the external diameter of the distal portion of the stimulation lead is at least the diameter of the suprarenal vein near its junction with the adrenal gland. The suprarenal vein has an internal diameter of between 3 and 8 mm, thus the external diameter of the stimulation lead in one embodiment is at least 3 mm, and can range from 3-16 mm in diameter. The distal spring geometry of the transvascular lead is configured to be placed within the intended anatomy for stimulation of the neural fibers that innervate the adrenal medulla, such as but not limited to the inferior vena cava (diameter range 10-25 mm), left or right renal vein (diameter range 8-16 mm), left or right suprarenal vein (diameter range 3-8 mm) and the inferior phrenic vein. In each stimulation lead, the external diameter may be oversized as much as 200% to allow the lead to conform to the size of the intended vessel as well as place just enough pressure on the vessel wall to allow the distal portion to be anchored without causing any vessel wall erosion.

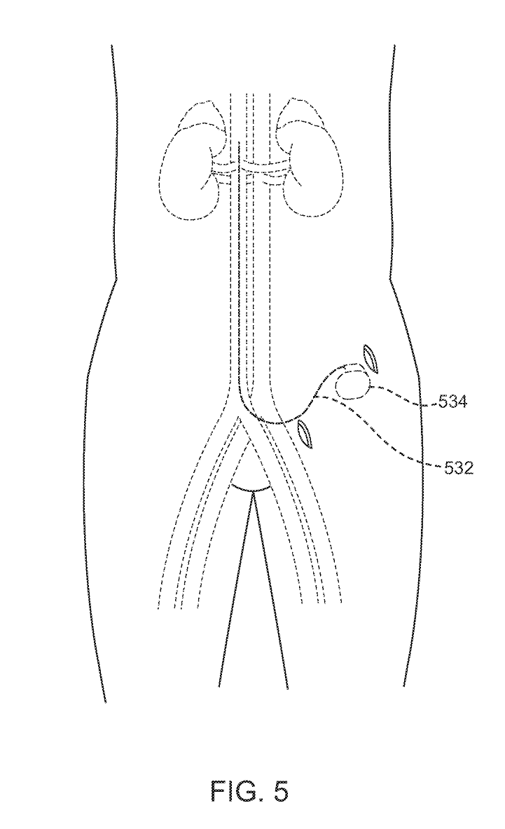

In one aspect of this embodiment, as shown in FIG. 5, the stimulation lead 532 can be connected to a neurostimulator 534 which can be implanted subcutaneously in the lower abdomen, by subcutaneously tunneling the lead to the neurostimulator. The stimulation lead is then connected and secured to the neurostimulator and the subcutaneous pocket is closed using standard wound closure methods.

In this embodiment, the neurostimulator may be implanted in the lower abdomen of the patient using a standard subcutaneous pocket, as shown in FIG. 5. Once the transvascular lead is placed within the targeted vessel and the guide wire, catheter and introducer are removed, the transvascular lead can be tunneled to the implant site of the neurostimulator. Once the proximal end of the lead is within the subcutaneous pocket where the neurostimulator will be implanted, the proximal portion of the lead is inserted into the neurostimulator and secured. The neurostimulator can be implanted into the subcutaneous pocket.

In one embodiment, the neurostimulator may include a rechargeable or primary cell battery that includes all the necessary electronics to support medium and/or short range telemetry for communication, battery recharging (in the case of the rechargeable system) and delivery of the therapeutic electrical stimulation waveform. The neurostimulator may be configured to deliver electrical stimulation in any of several forms well-known in the art, such as biphasic charge-balanced pulses, with parameters such as 1-1000 Hz or 5-50 Hz frequency, 0.04-2 ms pulse width; and 0.05-100 mA or 0.1-5 mA or 1-10 V amplitude. In addition, the electrical waveform can be controllable such that either anodic or cathodic stimulation may be applied, or such that anodic or cathodic stimulation may be selected for each electrode or combination of electrodes, including a conductive outer surface of the neurostimulator acting as an electrode. Electrical stimulation may be delivered continuously; intermittently; as a burst in response to a control signal; or as a burst in response to a sensed parameters, such as detected glucose levels or changes in cardiac rhythm such a increased or decreased heart rate. The electrical parameters may also be adjusted automatically based on a control signal or sensed parameters or by selection by the end user (patient). Furthermore the electrical parameters may be adjusted automatically based upon time of day and/or patient postural position or activity as sensed by an accelerometer or similar capability.

The methods of electrical stimulation as disclosed here may also be replaced with other forms of the neuromodulation, such as chemical, magnetic, optical, mechanical (including vibration) or a combination of two or more of these. In other embodiments the adrenal stimulator could be an implantable neurostimulator (including transvascular) or adrenal stimulation can be done using an external technique of stimulation of the adrenal gland including pulsed magnetic stimulation of the adrenal gland. Adrenal stimulation may be achieved by heating, cooling, ultrasound, radiofrequency energy, vibration, light or other radiated energy, or chemical input. In another embodiment, stimulation of the adrenal medulla may be accomplished by infusion of acetylcholine (Ach) or cholinergic agents into the adrenal parenchyma.

In other embodiments, the neurostimulator may be implanted in the upper, lateral buttock region, analogous to the typical position of an implanted spinal cord stimulator for the treatment of chronic pain, again using a subcutaneous pocket.

In another embodiment, the stimulation lead may be implanted within the suprarenal vein by accessing the azygos vein via one of the lower posterior intercostal veins, below the heart. The azygos vein provides an access point to the inferior vena cava that may allow for a less invasive approach than using the femoral vein as described above. This transvascular approach to implanting the stimulation lead is done by gaining venous access via a posterior intercostal vein below the heart, and then threading the lead into the azygos vein, then into the inferior vena cava and finally into the suprarenal vein. A transvascular system used in this embodiment can also contain an introducer and a series of catheters and guide wires as described above and used in a similar fashion.

Referring again to FIG. 4, a distal portion of the stimulation lead 428, which includes electrodes 432 for the delivery of the electrical stimulus and therapy, can be anchored and stabilized within the vessel using a predefined lead bias as described above. The stimulation lead can naturally take on the preformed bias within the vessel and apply a small amount of force to the vessel wall to anchor the lead in place. In one embodiment, up to 16 electrodes are positioned along the distal lead bias such that stimulation is directed toward the outer half of the lead. The electrodes of FIG. 4 may be equally spaced along the distal bias or have a custom spacing. The electrodes may be circumferential or directional on the lead body, for example.

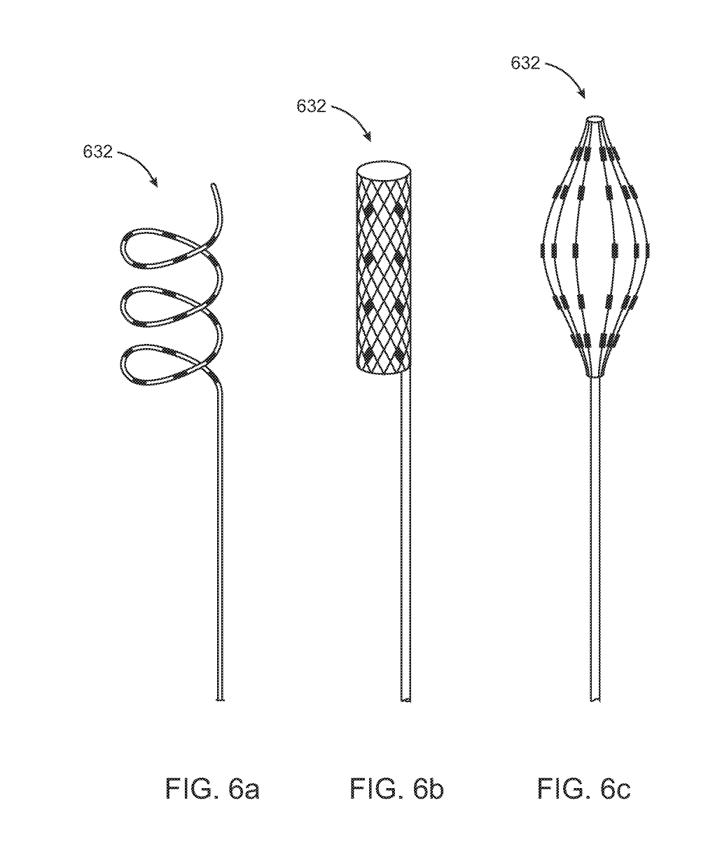

In some embodiments, the bias on the distal lead may be a corkscrew geometry, as shown in FIG. 6a. The bias can apply a predetermined amount of pressure on the vessel wall such that the lead is stable and the lead does not erode through the vessel wall. In other embodiments, the bias on the distal lead may have a loop or circular geometry, such that the loop is oriented perpendicular to the length of the vessel wall. The predefined bias may be created by creating an injection molding cast of the stimulation lead. The cast can then be injection molded with a standard biocompatible and flexible material, e.g. silicone, polyurethane or a combination thereof. The predefined bias is then the native geometry for the stimulation lead; however the stimulation lead can take other forms as required due to the flexibility of the lead material.

In other embodiments, the stimulation lead is delivered to the vessel using a flexible catheter system, such as described above. Once the catheter is correctly located within the target vessel, the stimulation lead can be inserted through the catheter. In one embodiment, the lead is not inserted over a guide wire, but instead inserted into the target vessel through a flexible catheter. The use of a guide wire may be done to help guide the flexible catheter to the intended vascular anatomy. Once the catheter containing the stimulation lead is in position, the catheter can then be retracted, leaving the stimulation lead in place.

In another embodiment, the distal portion of the stimulation lead may be deployed and anchored using balloon geometry, as shown in FIG. 6b, with many different spines in which one or more electrodes are placed. In yet another embodiment, the distal portion may have the geometry similar to a stent, as shown in FIG. 6c, again having one or more electrodes. In these embodiments, up to 16 electrodes 632 are positioned within the distal portion of the stimulation lead such that stimulation is directed toward the outer half of the lead. The electrodes 632 may be equally spaced or have a custom spacing. The electrodes may be configured to have a circumferential, rectangular, oval, or other well-known geometries. Additionally, the electrodes may be directional on the distal portion of the stimulation lead.

People with diabetes are not able to automatically maintain their blood glucose within safe physiological levels. They are taught how to compensate by monitoring their blood glucose levels several times a day. This invention uses stimulation of the adrenal gland to cause the release of epinephrine and other hormones that then cause glucose release from the liver to avoid hypoglycemia. However, in patients that experience frequent episodes of hypoglycemia, the presence of characteristic symptoms is diminished or gone, thus leaving these patients at risk for serious adverse effects of prolonged hypoglycemia. The control of the stimulation can be either open loop or closed loop; in the closed loop configuration the device can sense levels of glucose in the blood and automatically response with treatment.

In one embodiment of the open-loop system, a neurostimulator having a lead that interfaces with the adrenal gland is implanted in the patient. The neurostimulator produces electrical pulses suitable for causing epinephrine release from the adrenal gland. For example, charge balanced biphasic 1 mA pulses having a pulse width of 100 μsec can be produced by the neurostimulator in 30 second bursts of 5 Hz at a burst repetition interval of 5 minutes and applied to the adrenal gland. All parameters (amplitude, pulse width, burst duration, stimulation frequency, and burst repetition interval) may be adjusted to produce a low level of background epinephrine release suitable to provide protection against hypoglycemic events. Furthermore, the parameters may automatically vary depending for example on the time of day, or may be adjusted manually by the patient, caregiver or clinician. In some embodiments, the adrenal stimulator may be configured to apply continuous low level electrical stimulation to the neural fibers innervating the adrenal gland. A low level of stimulation may induce a constant slow release of CAs into the blood stream in very small amounts, similar to the use of a constant infusion pump. In another embodiment, the adrenal stimulator is capable of stimulation and causing the release of CAs on a scheduled basis. For example, the neurostimulator may be scheduled to deliver therapy at certain time frames through a 24 hr period, such that the amount of CAs in the blood stays at a relatively stable level throughout the day. In other embodiments the adrenal stimulator can be configured to communicate with an external patient remote, which give the patient the ability to turn on and off therapy, as well as adjust the stimulation parameters described above. The patient remote can be configured to communicate with the neurostimulator wirelessly using, for example, WiFi, Bluetooth, infrared or similar technology. In some embodiments, the patient can use the remote to turn on therapy as needed; for example, when the patient experiences symptoms characteristic of hypoglycemia.

In another embodiment, a closed loop system would take advantage of readings from a continuous glucose monitor. A continuous glucose monitor (CGM) is a small implantable device that measures the patient's glucose levels and telemeters that information to a display device that the patient uses to observe their current glucose levels and glucose level trends. Patient glucose levels telemetered from a CGM may be used by the adrenal stimulator to automatically stimulate the adrenal gland to release epinephrine when glucose levels are low or are trending low. By automatically monitoring glucose levels information from the CGM, the adrenal stimulator can maintain a safe level of glucose without the patient having to worry, and without stimulating the adrenal gland at a level that might result in glucose levels that are too high. Since there is a time delay between the stimulation of the adrenal gland and an increase in blood glucose, better performance may be achieved using predictive algorithms for adrenal stimulation.

In one such embodiment, the amount of adrenal stimulation may be determined using an algorithm, for example a proportional-integral-derivative control algorithm or a proportional derivative algorithm rather than determined by the absolute value of blood glucose. In addition, delivering the adrenal stimulation in boluses followed by monitoring blood glucose for the effect of the bolus can help avoid unintended overshoot of glucose levels and can be used by the adrenal stimulator for adaptive algorithms that self-adjust to the patients' current condition. In one embodiment, stimulation to the adrenal gland is delivered in bolus, for example, using stimulation parameters that cause the release of high concentration of catecholamines release (e.g. 40 Hz, for 1 second every 1 minute) and allowing time for the CGM to trend the effect of that quick bolus over that minute. Using the closed loop algorithm, the next bolus can be adjusted (increased or decrease in stimulus duration, amplitude, pulse width or frequency, or halting the next bolus) based on the effects of the last bolus or the last series of bolus'.

In other embodiments, the adrenal stimulator may also monitor cardiac parameters, especially cardiac rate, as an input to control or help control adrenal hormonal release that may increase heart rate. Monitoring heart rate provides input into a logic or controller block of the adrenal stimulator to adjust the adrenal modulation to maintain the heart rate within a target zone wherein the target zone may be fixed or may vary depending on time or other factors. In one embodiment, the adrenal stimulator is configured such that the controller block running an algorithm maintains a safe glucose range above a hypoglycemic level (for example above 70 milligrams/dL) while keeping the heart rate from increasing above a normal sinus rate (for example 75 beats per minute). In an alternative embodiment, the controller block can maintain a target heart rate (for example 80 beats per minute) without having high glucose levels (for example to not exceed 120 milligrams/dL). Such targeted heart rates could be automatically achieved on a regular basis over the course of a day by the implanted system to improve the metabolic state of the patient.

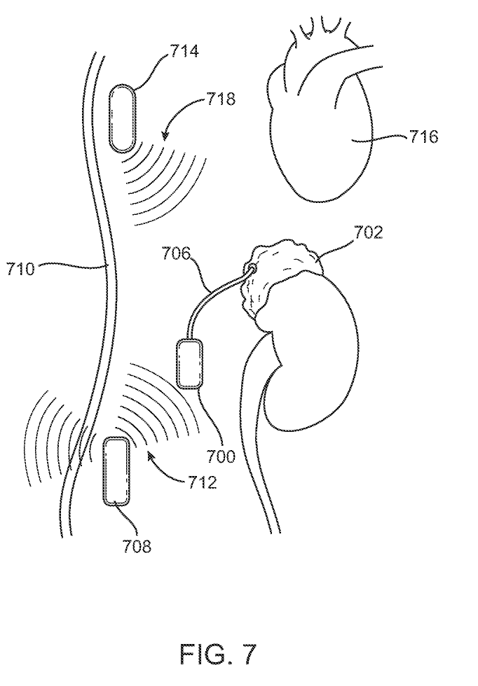

FIG. 7 illustrates a diagram of a system that includes an adrenal stimulator, CGM, and a heart rate monitor. The adrenal stimulator 700 is in communication with the adrenal gland 702 using a lead 706 that allows the adrenal stimulator 700 to stimulate the adrenal gland 702. Also implanted in the patient is a CGM 708 that communicates to a display device (not shown) outside the skin 710 of the patient. As shown here the CGM 708 communicates with the adrenal stimulator 700 by radio waves 712. In other embodiments, the communication between the CGM and the adrenal stimulator can be by a wire, or the CGM could be incorporated into the adrenal stimulator, or the communication between the CGM and the adrenal stimulator could be accomplished indirectly via an external device (not shown) in communication with both. Also implanted in the patient is a cardiac monitor 714 that detects the ECG from the patient's heart 716. As shown here the cardiac monitor 714 communicates between the cardiac monitor and the adrenal stimulator by radio waves 718. In other embodiments the communication between the cardiac monitor and the adrenal stimulator may be by wire, or the cardiac monitor can be incorporated into the adrenal stimulator, or the communication between the cardiac monitor and the adrenal stimulator could be accomplished indirectly via an external device (not shown) in communication with both.

In a further embodiment, the adrenal stimulator can be configured to allow the physician to prescribe therapeutic stimulation parameters such that different concentrations of CAs are released. For example, differential secretion of epinephrine and norepinephrine from the adrenal medulla is regulated by central and peripheral mechanisms. It is known that the CA concentrations released from the adrenal medulla during peripheral splanchnic nerve stimulation are altered by changes in stimulation frequency; thus, higher amounts of epinephrine are released with higher stimulation frequencies with continuous stimulation (20 Hz) in dogs (Mirkin 1961). Mirkin showed that using 20 Hz continuous stimulation produced higher concentration of epinephrine released than using 2, 5 or 10 Hz. The combination of distinct neural population recruitment via multiple electrodes on the stimulation lead and the use of different stimulus waveform parameters via the neurostimulator allow the physician to prescribe individualized therapy to each patient. In other embodiment, the adrenal stimulator can use different parameter sets to cause preferential release of certain concentrations of CA that preferentially effect glucose levels or heart rate for example, in response to the changes in glucose levels measured by the CGM or in changes in heart rate. For example, continuous stimulation between 2-20 Hz at an amplitude and pulse width know to cause electrical activation of the neural tissue or adrenal tissue is known to cause preferential release of epinephrine over norepinephrine in animal's models including, calves, sheep, cats and rats (Mirkin 1961, Edwards and Jones 1993), which will have a greater effect on glucose levels and heart rate. Or using stimulation at 40 Hz for 1 second, at 10 second intervals at an amplitude and pulse width know to cause electrical activation of the neural tissue or adrenal tissue is know to cause preferential release of norepinephrine over epinephrine in animal's models including, calves, sheep, cats and rats (Edwards and Jones 1993), which will have less of effect on glucose levels and heart rate. In an additional embodiment, the stimulation parameters used to cause preferential release of certain concentrations of CA may be used at specific times during the day. The optimization of these algorithms can individualize the patient care to the particular needs of that patient.

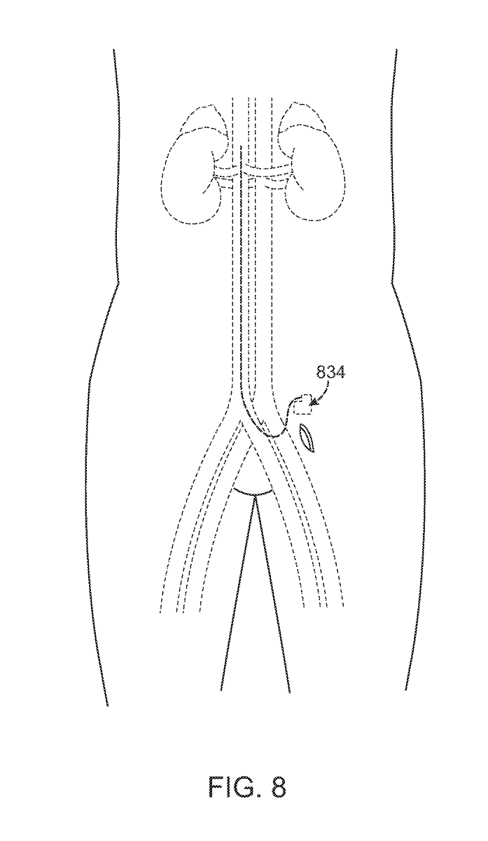

In one embodiment, the stimulation lead is placed as described above within the target vessel, but instead of tunneling the lead from the venous access site to the neurostimulator, a small externally powered neurostimulator 834 can be left at the site of the venous access, as shown in FIG. 8. In this embodiment a very small centimeter or millimeter scale neurostimulator 834 is implanted subcutaneously at the venous access site. This reduces excess trauma to the patient caused by tunneling the lead to a second incision site used to implant a larger neurostimulator, and may reduce the number of mechanical failures to the lead caused by body position and movements.

In this embodiment, the neurostimulator can be an inductively powered system that is configured to store programmable stimulation parameters, and has bi-directional telemetry to facilitate communication between the implanted neurostimulator and an external controller. The neurostimulator can include a custom ASIC, various passive components, and a secondary coil for radio frequency transfer of power and communication. The neurostimulator's custom ASIC may be configured to deliver electrical stimulation in any of several forms well-known in the art, such as biphasic charge-balanced pulses, with parameters such as 1-1000 Hz or 5-50 Hz frequency; 0.04-2 ms pulse width; and 0.05-100 mA or 0.1-5 mA, or 1-10 V amplitude. In addition, the electrical pulses can be controllable such that either anodic or cathodic stimulation may be applied, or such that anodic or cathodic stimulation may be selected for each electrode or combination of electrodes, including a conductive outer surface of the neurostimulator acting as an electrode. Electrical stimulation may be delivered continuously, intermittently; or as one or more bursts.

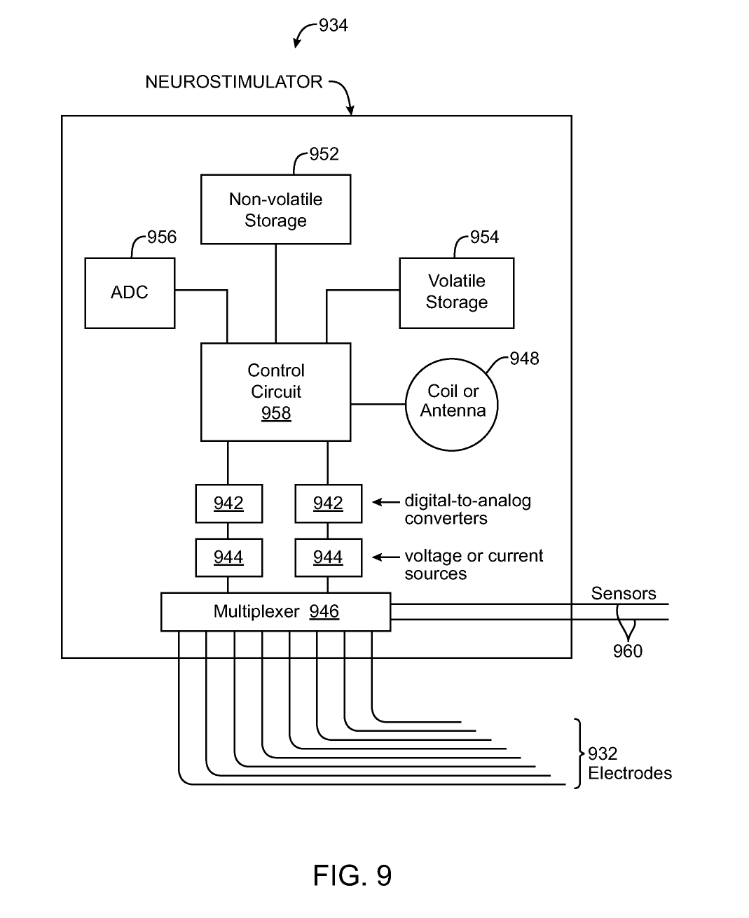

FIG. 9 shows an exemplary block diagram for a neurostimulator 934. Stimulation is delivered via one or more digital-to-analog converters 942 and voltage or current sources 944. A multiplexer 946 controls delivery of electrical current to electrodes 932. A coil or antenna 948 facilitates communication between a handheld controller and the neurostimulator. Non-volatile storage 952 and volatile storage 954 serve to record data related to stimulator function, or to store data that governs stimulator function. An analog to digital converter unit 956 may be included to facilitate measurement of internal or external voltages. A control circuit 958 such as a custom ASIC or microprocessor controls stimulation levels in response to transmitted signals.

The neurostimulator 934 of FIG. 9 may also include one or more sensors 960. These sensors may detect electrical signals (for example, electrocardiographic signals to determine heart rate), or the sensor might be an accelerometer to detect postural position or patient activity levels, or the sensor may detect substances such as circulating catecholamines using techniques well-known in the art such as optical or voltammetric detection. The control circuit 958 may transmit data acquired from these sensors to the handheld controller. The handheld controller may include one or more algorithms to automatically adjust stimulation parameters, including presence or absence of stimulation, frequency, pulse width, or amplitude according to the data received via the sensors or time of day. Additionally, a second coil or antenna may be include and customized to receive information from the CGM. For instance, the control circuit 958 may receive data via the CGM coil that indicates the blood glucose level is low (for example 70 micrograms/dL); then, using the automatic algorithm, the control circuit will trigger the appropriate type of adrenal stimulation based on programmed settings from the physician to increase circulating epinephrine and cause the release of glucose from the liver.

The handheld controller can be a hand held external, rechargeable, ergonomic, energy delivery device that transfers energy to the implanted stimulator with near field electromagnetic induction. The handheld controller can also be a communication system transferring information such as stimulation parameters to the implanted stimulator with bi-directional telemetry. The handheld controller can receive commands from an external programmer (such as a standard personal computer, with custom software configured to program the neurostimulator via the external controller), such as though a USB connection, for example. The handheld controller can communicate with the implanted stimulator once it is within close proximity to the stimulator. In one embodiment the handheld controller has features that allow it to deliver power along with sending commands to and receiving data from the neurostimulator.

In one embodiment, the controller communicates with the programmer through a USB cable connected between the controller and the programmer. When connected to the programmer, the controller enters a “pass through” mode in which all or some of its controls are disabled and it simply serves as a communication bridge between the PC and the stimulator.

In an alternate embodiment, the controller communicates with the programmer wirelessly using Wi-Fi, Bluetooth, infrared or similar technology.

The controller can include a power source such as batteries, a coil to inductively power the implanted adrenal stimulator and send/receive data, a microcontroller, firmware, wireless broadband card, supporting circuitry, an ergonomically shaped housing and various manual control features such as a therapy level adjustment knob or buttons, an off/on switch, and a display.

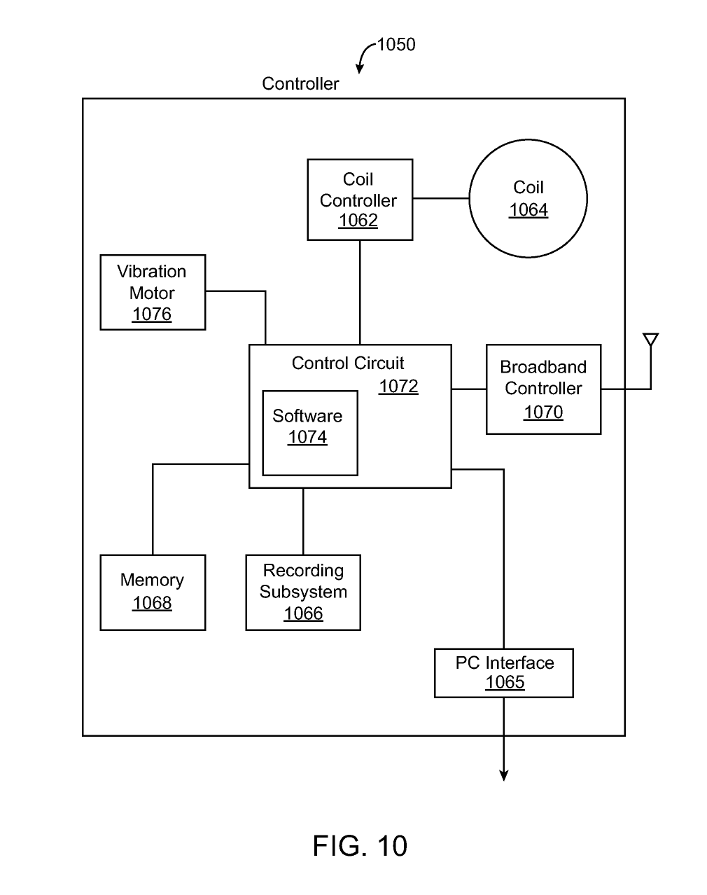

FIG. 10 shows an exemplary block diagram of a handheld controller 1050. A coil controller 1062 converts data to and from modulations in the inductive power signal, facilitating communication with the implanted stimulator. A PC interface 1065, such as a USB interface, is used to transmit and receive data to and from the programmer. A recording subsystem 1066 and memory 1068 provides logging of data describing stimulation delivery, such as timestamps of stimulation onset and data describing status or loss of communication with the implanted stimulator. These data may be uploaded wirelessly to a database using broadband controller 1070. A control circuit 1072, such as a microprocessor, executes software 1074.

When stimulation is initiated in this embodiment, the controller may optionally request data from the patient regarding disease severity or other symptoms. The controller will begin attempts to transmit and receive data with the implanted stimulator. The user may be provided feedback indicating strength and quality of the communication link. When stimulation is ongoing, control circuit 1072 and software 1074 acts to constantly monitor the implanted stimulator for events such as reset or electrical conditions such as when insufficient current is delivered. Actions taken by control circuit 1072 and software 1074 in response to these conditions may include re-initialization of the implanted stimulator, or notification provided to the patient or user, or logging of the event via the recording subsystem 1066.

In this embodiment, the therapy is provided to the patient in an on demand fashion. The neurostimulator in this embodiment is only powered when an external controller is positioned within close proximity and thus stimulation (and hence therapy) is only provided when the neurostimulator is powered. Thus a patient would use the external controller when they sense a hypoglycemic event starting to occur or occurring. The patient would discontinue therapy, thus removing the external controller from the vicinity of the implanted neurostimulator, when they sense the event dissipating.

In an alternative embodiment, the physician may prescribe the patient to use the external controller to provide therapy in a prophylactic manner in conjunction with on-demand therapy provided for each hypoglycemic event. In this embodiment, the patient applies periodic therapy throughout the day. This manner of therapy is similar to using a predefined therapy schedule as stated above within the use of the rechargeable or primary cell neurostimulator in an attempt to maintain a constant level of CAs in the blood stream, and thus maintaining the blood glucose level at a safe physiological range.

In another embodiment, a neurostimulator may be positioned in the vessel with the transvascular stimulation lead. The neurostimulator in this case may be positioned within the proximal vessel. In this case the neurostimulator may be designed to completely or at least partially anchor to the blood vessel in which the stimulation lead was implanted, thus anchoring the neurostimulator within the proximal, superficial anatomy. Additionally, in this embodiment the neurostimulator and the stimulation lead are one integral unit.

In an alternative embodiment the neurostimulator may be anchored using a deployable anchor system, such as a stent like mesh that expands to fit the diameter of the vessel upon retraction of the catheter system. In this embodiment the stent like mesh can be made of biocompatible metals, such as titanium, stainless steel, platinum, nitinol or polymeric or plastic materials. Alternatively the stent anchoring system may also act as a secondary receiving coil for the radio frequency powered neurostimulator as described above.

In other embodiments, the neurostimulator may be positioned within the distal vessel close to the area of deployment of the distal stimulation lead. In this embodiment the neurostimulator may be designed as a pod that again may be integral to the distal stimulation lead. In one embodiment the neurostimulator is designed to consist in part of a rechargeable battery and in other embodiments is designed to be powered using an external controller. Either embodiment would function as stated above for therapy delivery to the patient. In another embodiment, in which the distal lead is configured to have a stent like geometry as shown in FIG. 6c, the secondary coil, used for recharging or for supplying power and communication to the neurostimulator can be within the stent geometry and external to the neurostimulator. In yet another embodiment, the neurostimulator can be positioned between two separate lead biases, configured as described above except the neurostimulator has electrical connections to electrodes at both ends of the neurostimulator. In other embodiments transvascular stimulation may be done from the renal vein, inferior phrenic vein and or the inferior vena cava.

In the above embodiments, the neurostimulator is intended to apply a stimulus waveform to one or more neural structures that innervate the adrenal medulla including but not limited to the celiac plexus and ganglia, splanchnic nerves; greater, lesser and least, and other abdominal ganglia, such as the mesenteric and aorticorenal, or to the adrenal gland itself via a transvascular stimulation lead. In one embodiment, a transvascular stimulation lead is placed within the inferior vena cava at the level of the right adrenal gland. The transvascular lead is this embodiment is designed with a distal portion to fit within the diameter of the inferior vena cava, which has a diameter of between 10-25 mm in diameter. The stimulation lead may have an external diameter in its native, unstrained form of between 15 and 50 mm. Additionally, the distal portion of the lead can include at least 16 electrodes that may be equally spaced across the distal portion of the lead and in other embodiments may have a custom spacing and or alignment along the distal portion of the lead. For example, in one embodiment, the distal portion of the lead is designed to have a stent-like configuration that can be deployed through a flexible catheter. The electrodes on the stent are configured to be localized on the right posterior lateral quadrant of the inferior vena cava. The localization of the electrodes to the posterior lateral portion of the inferior vena cava can allow for localized stimulation of the neural fibers that are passing posterior to the vessel and directly innervate the right adrenal gland. This helps avoid potential unintentional stimulation of peripheral structures such as the descending vagus nerve trunks, aorta, and other peripheral structures.

Alternatively, in other embodiments, activation of the adrenal medulla chromaffin cells may be done by direct stimulation of the neural fibers that innervate the chromaffin cells and cause the release of CAs. In many cases the neural fibers that innervate the adrenal gland travel next to or on the arterial supply. The adrenal glands are supplied by many arterial branches from the descending aorta including but not limited to the renal artery, inferior suprarenal artery, middle suprarenal artery, superior suprarenal artery and the inferior phrenic artery. Many, if not all of the neural fibers innervating the adrenal gland travel with or in very close proximity to these arterial supplies.

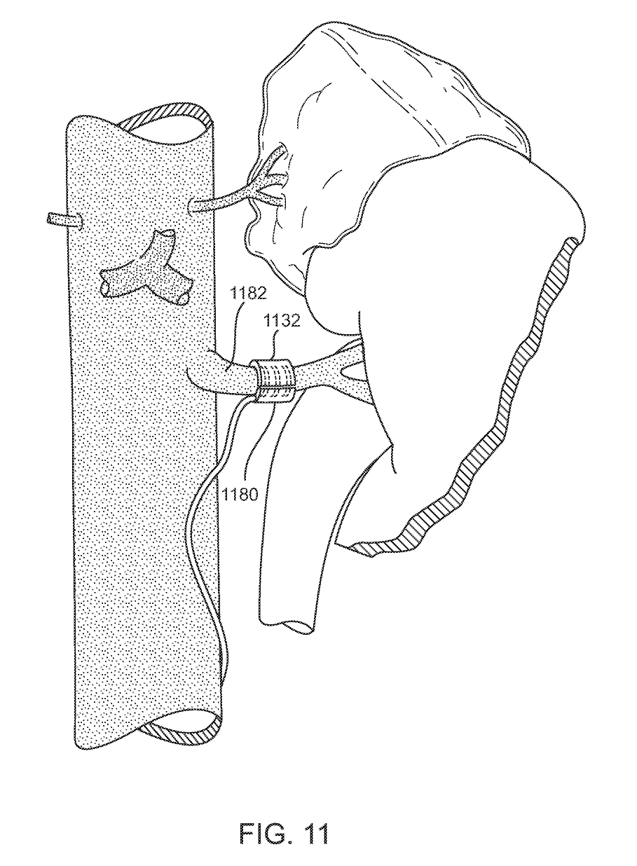

In one such embodiment, shown in FIG. 11, an electrical waveform may be applied to the neural fibers innervating the adrenal medulla through one or more electrodes 1132 contained within a neural cuff 1180 designed to encircle the renal artery and stimulate the neural fibers that travel along the renal artery 1182 and innervate the adrenal medulla. The neural cuff may be implanted using standard open, laparoscopic or endoscopic surgical techniques to expose the adrenal gland and the surrounding vasculature. Each electrode can be embedded within the cuff and placed on the inner wall of the lead such that the electrode either directly contacts the neural fibers along the renal artery or is placed within a few millimeters or less of the neural fibers. The neural cuff may have a cylindrical geometry with a split running the length of the cuff portion of the lead to facilitate placement of the cuff lead around the artery of interest. Additionally, the neural cuff may be made from a biocompatible, flexible and soft material that may include but is not limited to silicone, polyurethane, other polymer and plastic materials, or any combination of these materials. In another embodiment the length of the distal cuff lead is between 12 and 25 mm in length, more specifically 18 mm in length and having an internal diameter that corresponds with the external diameter of the renal artery (4-8 mm).

In one embodiment the cuff comprises at least three electrodes that extend along the inner circumference for at least 270 degrees and have a width of between 0.5-2 mm. In other embodiments, the cuff consists of at least three electrodes positioned in a ring around the inner circumference of the cuff and has at least three such rings positioned along the length of the cuff lead. Each electrode in this embodiment may be between 0.5 and 4 mm in length and 0.5 to 2 mm in width. In other embodiments, the multiple electrodes of multiple shapes and sizes can be positioned around the inner surface of the neural cuff. In each of the above embodiments, each electrode can be made out of a standard biocompatible and inert metal that is well known in the art, such as platinum, iridium, stainless steel, gold, other metals, or any combination of these materials.

In other embodiments, the neural cuff may be placed on or around one or more arteries innervating the adrenal gland, including but not limited to the renal artery, superior suprarenal artery, middle suprarenal artery, or the inferior suprarenal artery. The renal artery as described above has an external diameter of between 4 and 8 mm, additionally the suprarenal arteries (superior, middle and inferior) have an external diameter between 0.5 and 5 mm. Thus a neural cuff may be designed to have an internal diameter of 0.5 to 8 mm. In other embodiments, the neural cuff may only have one size, which is adjustable to the needed diameter of the vessel of interest. In one embodiment, this is done by using a spiral cuff design that has multiple turns and allows the cuff to be implanted on a range of different vessel diameters. In another embodiment, the neural cuff has multiple spiral cuffs connected together through one spine. Each of the spiral cuffs contains one or more electrodes.

The neural cuff is connected to an implanted neurostimulator through a lead, and the neurostimulator may be implanted at a location near the posterior lateral buttock region or in the lower abdomen using a standard subcutaneous pocket. As described above, the neurostimulator can be designed to have a rechargeable or primary cell battery, or be powered from an external controller. Additionally, once the adrenal stimulator is implanted in the retroperitoneal space, energy to power the pulse generator may be obtained from sources of energy available in or near the body, using energy-harvesting devices or methods well-known in the art; for example, antennas, photovoltaic cells, rotating-mass kinetic generators, piezoelectric generators, or thermoelectric generators. Sources of energy available in or near the body may include acceleration, temperature gradients present in the body or at the surface of the body; light available at or near the surface of the body; ambient electromagnetic or other radiation; or chemical energy present in substances such as blood glucose.

As described above, the adrenal stimulator may be configured to deliver electrical stimulation in any of several forms well-known in the art, such as biphasic charge-balanced pulses, with parameters such as 1-1000 Hz or 5-50 Hz frequency, 0.04-2 ms pulse width; and 0.05-100 mA or 0.1-5 mA or 1-10 V amplitude. In addition the electrical pulses can be controllable such that either anodic or cathodic stimulation may be applied, or such that anodic or cathodic stimulation may be selected for each electrode or combination of electrodes, including a conductive outer surface of the neurostimulator acting as an electrode. Electrical stimulation may be delivered continuously, intermittently; or as one or more bursts. Non-pulsatile waveforms including sinusoidal, near-sinusoidal, square, triangular, or sawtooth waves at frequencies of 1-100 Hz may also be used. Also as described above, the adrenal stimulator can be configured to receive information from integral or external sensors, such as an external CGM or an integral heart rate monitor. Therapy can also be applied as stated above either continuously, at scheduled intervals over a 24 hour period or on demand by the patient.

A standard endoscopic, laparoscopic or open surgical technique may be used to place the neural cuff lead around the artery of interest that supplies the adrenal gland and carries the neural innervation to the adrenal medulla. In one embodiment, the neural cuff lead is implanted using a standard endoscopic retroperitoneal approach to the adrenal gland and surrounding neuro-vascular tissue as described by Bonjer (Bonjer, Sorm et al. 2000). In another embodiment, the neural cuff lead projects from a neurostimulator located in the retroperitoneal space, and is implanted around the superior suprarenal artery. In this embodiment, it is desirable that the leads are mechanically compliant and fatigue resistant in order to prevent trauma to the adrenal tissue and to avoid breakage with normal body movements (similar to a conventional cardiac or spinal cord stimulator lead). In other embodiments, stimulation to cause the release of CAs from the adrenal medulla may be done by stimulating the chromaffin cells within the adrenal medulla or by stimulating the pre-ganglionic sympathetic fibers within the adrenal medulla that synapse onto the chromaffin cells. Stimulation of the adrenal gland chromaffin cells or the fibers that synapse onto the cells may be done by applying a stimulus waveform to the body of the adrenal gland directly. Alamo et al and Wakade (Wakade 1981; Alamo, Garcia et al. 1991) have shown that a stimulus applied to the exterior surface of the adrenal gland can affect the adrenal medulla, causing CA release.

In one embodiment, one or more electrodes are anatomically placed around the adrenal cortex and a stimulus waveform is applied to cause the release of CAs for the treatment of hypoglycemia. In this embodiment, a minimally invasive standard endoscopic retroperitoneal approach is used to surgically expose the adrenal gland and an externally applied surface stimulation lead is placed near or in contact with the outer membrane of the adrenal cortex. The lead can be configured to have the geometry resembling a Y, having three individual fingers that are configured to wrap around the adrenal gland along the long axis of the gland. The adrenal gland is approximately 4-6 cm in length, usually 2-3 cm in width and 0.2-0.6 cm thick and is covered by a tight membrane. Using endoscopic instruments, the Y type lead can be placed around the outer membrane of the adrenal gland. Each finger of the Y type lead is configured to have one or more surface electrodes for delivery of the stimulus waveform. The Y type lead is designed to have three flexible members that extend from a central point at (for example) 120 degrees angles from each other and extending from the central point 1-5 cm in order to fully encompass the adrenal gland. Each flexible member may contain one or more electrodes that are shaped and composed similarly to electrodes described in this invention above. Additionally, the native orientation of the flexible finger-like members is in closed fist state, in which each finger is naturally curved such that the inner radius of the curve is approximately the width of the adrenal gland (2-3 cm). A malleable stylet may be provided such that during implantation of the Y stimulation lead, the fingers can be opened and the lead may be placed around the outer member of the adrenal gland. Once the correct placement is achieved, the stylet can be removed and the lead will assume its natural orientation and curl around the adrenal gland.

In another embodiment, the Y stimulation lead is configured to have penetrating elements that penetrate the cortex of the adrenal gland when positioned, and at least partially place one or more electrodes within the adrenal medulla. The penetrating elements in this embodiment may be made out of silicon with one or more electrodes spaced along the length of the element, thus allowing for the positioning of electrodes across the adrenal gland. In other embodiments, the elements may be made from but not limited to silicone, polyurethane, polymers, plastics or any combination thereof. In one embodiment each, penetrating element has a length of approximately 0.1 to 0.5 cm. In another embodiment, the Y stimulation lead may have more than 3 flexible members extending from a central point, and each member may be configured to have one or more surface electrodes or penetrating elements with one or more electrodes or any combination of either configuration.

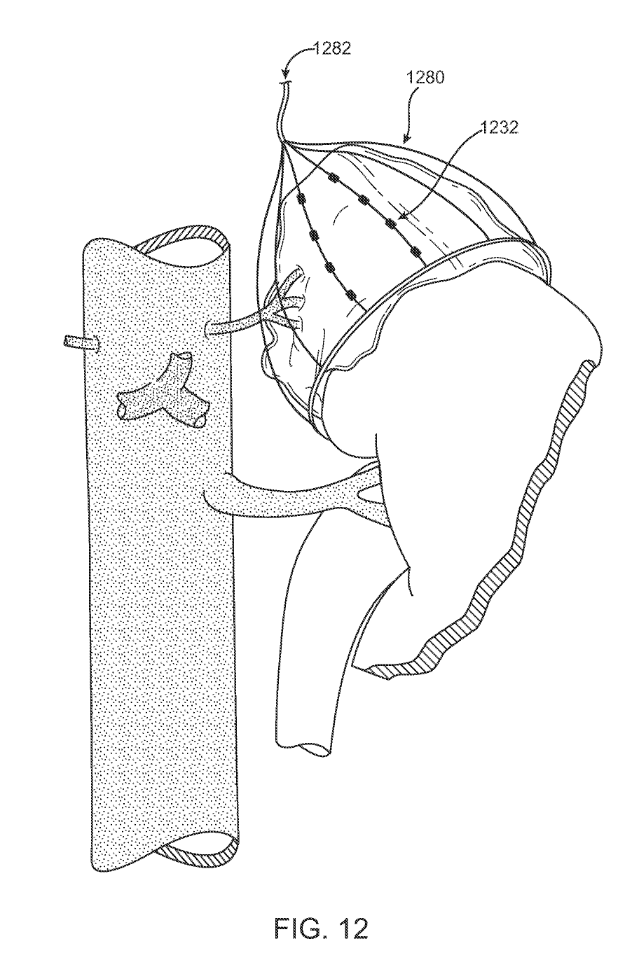

In another embodiment, the distal end of a stimulation lead is configured in the form of a sac, partial sac, net, or hemisphere. In one embodiment, as shown in FIG. 12, a lead is connected to a distal sac 1282 configured to surround or partially surround the adrenal gland. The sac 1282 is configured to contain one or more flexible spines 1280 that allow the sac to retain this shape once deployed around the adrenal gland. These spines can be made of memory retention material such as but not limited to Nitinol. In one embodiment, each spine within the sac may contain one or more stimulating electrodes 1232 which may be disposed on the inner surface of the spine so as to contact the gland. The distal end of the lead may be constructed of an elastic or compliant material, including polymer mesh, to promote contact between the electrodes and the gland. A mechanism, such as a drawstring, may be provided to secure the distal end of the lead around the gland.

In another embodiment, the distal end of the lead is configured in the form of a sac, partial sac, net or hemisphere and contains a port that extends to an implantable reservoir along the length of the lead. The implantable reservoir may be an implantable drug pump that is programmable. In one such embodiment, stimulation of the adrenal medulla may be accomplished by the infusion of acetylcholine (ACh) or other cholinergic agents into the distal lead sac, partial sac, net or hemisphere to stimulate the chromaffin cells to release CAs. The implantable reservoir can be configured much like the neurostimulator in that it can apply a continuous small amount of ACh in order to stabilize the amount of CAs in the blood stream, release a known amount on a scheduled basis or on-demand boluses by the user when an hypoglycemic event. In other embodiments, a combination device may be used in which the stimulation device is configured to have both a neurostimulator and a reservoir.

In further embodiments, indirect stimulation of the adrenal medulla to cause the release of CAs for the treatment of hypoglycemia may be accomplished by stimulating various areas of the sympathetic nervous system that innervate the adrenal medulla. These areas of the sympathetic nervous system include but are not limited to the splanchnic nerves (greater, lesser, least), peripheral ganglia (e.g. celiac, mesenteric), and the thoracic sympathetic trunk. Direct electrical stimulation to these sympathetic neural structures may indirectly cause the release of CA from the adrenal medulla for the treatment of hypoglycemia. Direct stimulation of these neural structures can be done using methods similar to those described above using a neural cuff electrode or using traditional linear array electrodes. In one embodiments, a neural cuff electrode is implanted directly on the splanchnic nerves, including the greater, lesser and least. The neural cuff may be implanted using standard open, laparoscopic or endoscopic surgical techniques to expose the abdominal neural plexus. In other embodiment, a linear array electrode can be used to directly simulate the thoracic sympathetic trunk, or one or more of the peripheral sympathetic ganglia that innervate the adrenal gland, including but not limited to the celiac, mesenteric and aorticorenal ganglia. The linear array electrode can be implanted using standard techniques, and in one embodiment, the linear array electrode can be surgically placed next to the thoracic sympathetic trunk using standard techniques. In one embodiment, the linear array electrode includes a distal portion that is cylindrical in cross section and has a diameter between 1-3 mm. The linear array electrode also includes one or more stimulation electrodes, as describe elsewhere in this invention.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A method of treating a disorder, comprising:
placing a stimulation lead on neural tissue in communication with an adrenal gland; and
delivering an electrical waveform from a neurostimulator to the stimulation lead to release catecholamines from the adrenal gland to treat hypoglycemia.

2. The method of claim 1 wherein the stimulation lead is placed on or in an inferior vena cava.

3. The method of claim 1 wherein the stimulation lead is placed on or in a left or right renal vein.

4. The method of claim 1 wherein the stimulation lead is placed on or in a left or right suprarenal vein.

5. The method of claim 1 wherein the stimulation lead is placed on or in an inferior phrenic vein.

6. The method of claim 1 wherein the stimulation lead comprises a coiled shape.

7. The method of claim 1 wherein the stimulation lead comprises at least one electrode.

8. The method of claim 1 wherein the stimulation lead is placed on or near a thoracic sympathetic trunk.

9. The method of claim 1 wherein the stimulation lead is placed on or near a splanchnic nerve.

10. The method of claim 1 wherein the neurostimulator is implanted in a lower abdomen of the patient.

11. The method of claim 10 wherein the stimulation lead is tunneled from a venous access site to the neurostimulator.

12. The method of claim 1 wherein the neurostimulator is implanted at a site of venous access.

13. The method of claim 1 wherein the neurostimulator is implanted within a vessel.

14. The method of claim 1 wherein the delivering step further comprises delivering the electrical waveform from the neurostimulator to the stimulation lead by positioning an external controller in close proximity to the neurostimulator.

15. The method of claim 1 wherein the stimulation lead comprises a neural cuff configured to stimulate nerves innervating the adrenal gland.

16. The method of claim 15 wherein the neural cuff is approximately 12 to 25 mm in length.

17. The method of claim 15 wherein the neural cuff has an internal diameter that corresponds with an external diameter of a renal artery.

18. The method of claim 15 wherein the neural cuff comprises electrodes that extend along an inner circumference for at least 270 degrees.

19. The method of claim 1 wherein the stimulation lead is placed around an adrenal cortex and is configured to stimulate an adrenal medulla.

20. The method of claim 19 wherein the stimulation lead has a geometry resembling a Y and comprises at least one electrode.

21. The method of claim 19 wherein the stimulation lead comprises penetrating elements configured to penetrate the cortex of the adrenal gland.

22. The method of claim 1 wherein the stimulation lead comprises a net configured to at least partially surround the adrenal gland.

23. The method of claim 22 wherein the net comprises a plurality of flexible spines configured to allow the net to retain its shape upon deployment around the adrenal gland.

24. The method of claim 22 wherein the net further comprises a drawstring configured to secure the net around the adrenal gland.

25. The method of claim 22 wherein the stimulation lead comprises at least one electrode.

26. A method of treating a disorder in a patient, comprising:
placing a stimulation lead on neural tissue in communication with an adrenal gland;
measuring a glucose level of the patient with a continuous glucose monitor; and
automatically applying an electrical waveform from the stimulation lead to the neural tissue when the glucose level is low to release catecholamines from the adrenal gland to treat hypoglycemia.

27. The method of claim 26 wherein the stimulation lead comprises at least one electrode.

28. The method of claim 26 wherein the stimulation lead is placed on or near a thoracic sympathetic trunk.

29. The method of claim 26 wherein the stimulation lead is placed on or near a splanchnic nerve.

30. The method of claim 26 further comprising monitoring a heart rate of the patient with a cardiac monitor.

31. The method of claim 30 wherein the electrical waveform is automatically applied based on the glucose level and the monitored heart rate of the patient.

32. A system for stimulating an adrenal gland, comprising:
a continuous glucose monitor configured to measure a patient glucose level;
a stimulation lead; and
an adrenal stimulator in communication with the continuous glucose monitor and the stimulation lead, the adrenal stimulator configured to apply an electrical waveform to the stimulation lead in response to the measured patient glucose level.

33. The system of claim 32 further comprising a cardiac monitor configured to measure a cardiac parameter.

34. The system of claim 32 wherein the stimulation lead comprises a coiled shape.

35. The system of claim 32 wherein the stimulation lead is sized and configured to be implanted on neural tissue in communication with an adrenal gland.

36. The system of claim 32 wherein the stimulation lead is a neural cuff.

37. The system of claim 32 wherein the stimulation lead is a sac.

38. The system of claim 32 wherein the stimulation lead comprises at least one electrode.

39. The system of claim 32, wherein the adrenal stimulator is in radio communication with the continuous glucose monitor.
